(19) United States
(12) Patent Application Publication
(10) Pub. No.: US 2018/0318291 A1
(43) Pub. Date: Nov. 8, 2018

(54) USE OF AMINOTHIAZOLE MYD88 SPECIFIC INHIBITOR TJM2010-5

(71) Applicant: WUHAN INNAMUNE PHARMACEUTICAL INC. LTD., Wuhan (CN)

(72) Inventor: Ping ZHOU, Wuhan (CN)

(21) Appl. No.: 15/773,367

(22) PCT Filed: Nov. 16, 2015
(86) PCT No.: PCT/CN2015/094747

§ 371 Date: May 3, 2018

(30) Foreign Application Priority Data
Nov. 5, 2015 (CN) 201510753839.7

(51) Int. Cl. A61K 31/496; A61P 9/10; A61P 37/02; A61P 29/00

(52) U.S. Cl. CPC A61K 31/496; A61P 29/00; A61P 37/02; A61P 9/10

(57) ABSTRACT

MyD88 inhibitor can be used to prevent, for example, post myocardial infarction ischemia reperfusion injury, post severed limb replantation ischemia reperfusion injury and post transplantation ischemia reperfusion injury, and to play important role in production of organ preservation solution, cell preservation solution etc. Furthermore, results of in vitro experiment show that MyD88 inhibitor TJM2010-5 can effectively reduce the level of inflammatory factors in graft through inhibiting the pathway, which means that it is closely related to the generation of inflammatory factors, therefore, the TJM2010-5 can become the effective method for treating of various inflammations, prevent the vicious circle and vicious transformation of chronic inflammation, and prevent and treat inflammatory cancerization etc.

### BACKGROUND OF THE INVENTION

### Technical Field of the Invention

Provided in the invention is the use of an aminothiazole medicament, i.e. the aminothiazole MyD88 specificity inhibitor TJM2010-5, in particular the preparation of immunesuppressant and immuneregulator.

### Background Art

The medical evidences have shown that the inhibitory regulation of immune system is critical for treatment of various diseases, such as the treatment of organ transplant rejection, autoimmune disease, inflammatory disease and tumor, ischemia reperfusion injury etc.

Although it has been known that the immune system consists of innate immunity and acquired immunity, the later one has always been considered as the main research object and intervention target of immunity, as it has a highly specific recognition function and an efficient reaction effect. Traditionally, the immune reaction is considered as the process that NF-κB, activated by the first and second stimulatory signals of acquired immunity, enters the cell nucleus to initiate the transcription, and the cells begin to synthesize and secrete various inflammatory cytokines which trigger a serial of subsequent immune reactions. And the current anti-refection medicaments are all targeted to acquired immunity. The innate immunity has been ignored, as it is considered as the natural protective barrier of the body against infection of virus and bacteria, and alien biological invasion etc. However, a lot of researches conducted in recent years have shown that the innate immune system plays a more important role in transplantation immunity, autoimmune disease and ischemic injury etc. And the tool-like receptor (TLR) in the system plays the most import role and is highly stressed. It has been discovered that TLR has at least 14 subtypes, which are mainly distributed on immune cells, such as APC, and except for TLR3, all these subtypes submit signals through myeloid-differentiation protein 88 (MyD88) molecule. The numerous researches has shown that various endogenic and ectogenic risk factors will stimulate the TLR in innate immune system, and the stimulate signal transmitted through the key molecule MyD88 will activate the kinases in signal pathway, such as IRAKs, TRIF, TRAM/TRIF, IRF7 etc., where, IRAK will finally activate NF-κB thus to incur the same subsequent immune reaction process as that described above; and other pathway will activate the inflammatory reaction through regulating inflammatory factor (such as IFN- and IFN-) and its gene expression. For this reason, MyD88 is the key molecular node of innate immunity, and the main reaction of innate immune system can be prevented by blocking MyD88, thus to generate an extensive and strong immune inhibitory effect. The inhibitory effect is higher than that of the traditional medicaments of acquired immunity targeted to NF-κB.

### SUMMARY OF THE INVENTION

The present purpose aims to provide the aminothiazole small molecule compound TJM2010-5 as the MyD88 specificity inhibitor, to treat various immune-related diseases by utilizing its inhibitory effect on MyD88 molecule in innate immune system. The basis for carrying out the specific technical scheme of the invention is that the application of TJM2010-5 in prevention of transplant rejection and graft-versus-host reaction (GVHD), autoimmune disease, ischemia reperfusion injury; organ and tissue cells preservation; prevention and treatment of vicious circle and vicious transformation (canceration) of chronic persistent inflammation, and endoxemia is firstly proposed in the invention by utilizing its inhibitory effect on MyD88 molecule in innate immune system.

The MyD88 protein described here is consisted by two domains: the TIR domain (toll/IL-1 receptor domain) and DD domain (death domain), where the TIR domain is the material basis for MyD88 to generate auto-homodimer, through which the kinases in downstream, such as IRAK1 or IRAK4, are activated. The MyD88 specificity inhibitor TJM2010-5 is synthesized according to the analysis on topological structure of TIR domain of MYD88. The inhibitor TJM2010-5 can have specific binding in the TIR domain of MyD88, intervene the function of TIR domain of MyD88, and prevent the generation of homodimer, to prevent the activation of MyD88, thus to block the conduction of MyD88 pathway, which will prevent the activation of kinases in downstream, such as IRAKs, TRIF, TRAM/TRIF, IRF7 etc. For this reason, the IRAK cannot activate NF-κB, and other kinases cannot activate other pathways, including such inflammatory factors as IFNs, which will prevent the inflammatory reaction. Therefore, it plays an important role in treatment of related inflammations and immune system diseases.

The molecular structure of aminothiazole MyD88 specificity inhibitor described in the invention as follows:

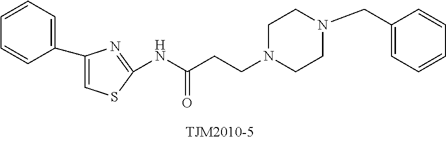 [SMILES: O=C(CCN1CCN(Cc2ccccc2)CC1)Nc1nc(-c2ccccc2)cs1]

3-(4-benzylpiperazin-1-yl)-N-(4-phenylthiazol-2-yl) propanamide

The MyD88 specificity inhibitor TJM2010-5 has small molecule and stable structure, and can pass through cell membrane. It can be administrated in vivo and vitro simultaneously.

A serial researches on TLR/MyD88 carried out by the designer of the invention proved that TLR plays an important role in immunity, and the research on mice had been treated by MyD88 gene knockout proved that the blocking of MyD88 molecule can induce and maintain the transplantation immune tolerance. With the cooperation of the pharmacy team, the designer of this invention manufactured and synthesized the MyD88 specificity inhibitor: code of TJM2010-5 after repeated screening in later researches. The small molecule compound can achieve specific binding as the active sites in its structure can match with the key active sites of MyD88 molecule, so it can inhibit the corresponding signal conduction of MyD88 through competitive binding. Therefore, MyD88 specificity inhibitor TJM2010-5 is applied to prevent transplant rejection and graft-versus-host reaction (GVHD), autoimmune disease, ischemia reperfusion injury; and to prevent and treat chronic inflammation and vicious transformation (canceration) of colon, liver and other organs, and endoxemia etc., which is pioneering in application of such small molecular substances, and provide a possible new medicament therapy for various innate immune related diseases.

The aminothiazole MyD88 specificity inhibitor TJM2010-5 provided in this invention is used as inhibitor of NF-κB and various inflammatory factors in preparing immunoregulatory medicaments.

The aminothiazole MyD88 specificity inhibitor TJM2010-5 provided in this invention is used in preparing medicaments for treating and reducing transplant rejection, and inducting and maintaining transplantation immune tolerance, such as preventing and treating transplant rejection, inducing transplantation immune tolerance and preventing graft-versus-host reaction (GVHD) etc.

The aminothiazole MyD88 specificity inhibitor TJM2010-5 provided in this invention is used in preparing medicaments for treating various inflammations, chronic inflammatory diseases and the related inflammation-associated tumors.

The aminothiazole MyD88 specificity inhibitor TJM2010-5 provided in this invention is used in preparing medicaments for treating various autoimmune diseases, such as diabetes mellitus type I, multiple sclerosis and lupus erythematosus etc.

The aminothiazole MyD88 specificity inhibitor TJM2010-5 provided in this invention is used in preparing protection agents for ischemia reperfusion injury, such as preparing medicaments for preventing and treating the post myocardial infarction ischemia reperfusion injury, post severed limber plantation ischemia reperfusion injury, and post transplantation ischemia reperfusion injury, as well as preparing organ preservation solution, tissue preservation solution and cell preservation solution etc.

The aminothiazole MyD88 specificity inhibitor TJM2010-5 provided in this invention is used in preparing prevention and cure medicaments for endoxemia and septicopyemia.

The aminothiazole MyD88 specificity inhibitor TJM2010-5 provided in this invention is used in preparing therapeutic medicaments for colitis and colorectal cancer. TJM2010-5 is used to block the inflammatory reaction and vicious circle of inflammation at the initiation site of inflammatory reaction as the inhibitor of immune reaction initiation site, and to treat various chronic intractable colitis and to prevent its vicious transformation (cancer, inflammation—associated cancer) by changing the types and proportion of inflammatory cells, oncogenic inflammatory cells, macrophages etc. in inflammatory focus, such as chronic colitis and its canceration (colorectal cancer), chronic hepatitis hepatocirrhosis and related liver cancer, chronic bronchitis and lung cancer, chronic atrophic gastritis, gastric ulcer and gastric cancer etc.

Beneficial effects of the invention: in this invention, the compound TJM2010-5 was applied as the MyD88 specificity inhibitor in the experiments, which proved that TJM2010-5 has significant effects in preventing transplant rejection and inducing immune tolerance, treating various inflammatory reactions, preventing and treating ischemia reperfusion injuryetc etc., and it will become an efficient immunosuppressant, or transplantation tolerance inducer, or transplantation tolerance maintenance agent, anti-inflammatory medicament and immunoregulator. This new compound can effectively inhibit the expressions of CD80, CD86, thus to prevent the maturity of DC cell, while the maturity of DC cell has been proven to be one of the key steps in development of various autoimmune diseases, such as autoimmune cardiomyopathy, experimental autoimmune uveitis, diabetes mellitus type I, multiple sclerosis and lupus erythematosus etc. Therefore, MyD88 inhibitor TJM2010-5 can be used for treatment of such diseases. The blocking of MyD88 pathway has significant effect on protection against ischemia reperfusion injury, therefore, MyD88 inhibitor can be used to prevent, for example, post myocardial infarction ischemia reperfusion injury, post severed limb replantation ischemia reperfusion injury and post transplantation ischemia reperfusion injury, and to play important role in production of organ preservation solution, cell preservation solution etc. Furthermore, results of in vitro experiment show that MyD88 inhibitor TJM2010-5 can effectively reduce the level of inflammatory factors in graft through inhibiting the pathway, which means that it is closely related to the generation of inflammatory factors, therefore, the TJM2010-5 can become the effective method for treating of various inflammations, prevent the vicious circle and vicious transformation of chronic inflammation, and prevent and treat inflammatory cancerization etc.

### BRIEF DESCRIPTION OF THE DRAWINGS

FIG. 1a is the survival curve showing the effect of TJM2010-5 in preventing heart rejection.

FIG. 1b is the survival curve showing the effect of TJM2010-5 in preventing skin graft rejection.

FIG. 2a shows the curative effect of TJM2010-5 on GVHD, where, FIG. 2a-A shows the differences among weights of TJM2010-5 treatment group and control group, FIG. 2a-B shows the GVHD levels of TJM2010-5 treatment group and control group, and FIG. 2a-C shows the survival rates of TJM2010-5 treatment group and control group.

FIG. 2b shows the effect of TJM2010-5 combined with MR1 in treating acute GVHD in mice, where, FIG. 2b-A shows the weight differences, FIG. 2b-B shows the differences among GVHD levels of the groups, and FIG. 2b-C shows the differences among survival rates of the groups.

FIG. 2c shows the difference among general performances and GVHD levels of each tissue sections of the groups. Where, FIG. 2c-A shows differences among appearance conditions of mice, FIG. 2c-B shows the differences among skin tissue sections of the groups, FIG. 2c-C shows the differences among liver tissue sections of the groups, FIG. 2c-D shows the differences among intestine tissue sections of the groups, and FIG. 2c-E shows the differences among spleen tissue sections of the groups (where, 1 refers to the control group, 2 refers to the single treatment group of TJM2010-5, 3 refers to the single treatment group of MRI, and 4 refers to the combination treatment group of TJM2010-5 and MR1).

FIG. 3a shows that TJM2010-5 can dose-dependently reduce the activation of T cells.

FIG. 3b shows that the LPS, CPG, and cardiac tissue homogenate vehicle can stimulate DC, and TJM2010-5 can down-regulate CD80 expression.

FIG. 3c shows that TJM2010-5 can dose-dependently down-regulate CD80 expression on DC surface.

FIG. 3d shows that TJM2010 can dose-dependently down-regulate CD80 expression on macrophage cell.

FIG. 4 is the curve showing the reduction of diabetes incidence rate by TJM2010-5.

FIG. 5 is the histogram showing effect of TJM2010-5 on lymphocyte subpopulation in the transplant receptor.

FIG. 6 is the survival curve showing improvement effect of TJM2010-5 on IRI survival rate of mice kidney.

FIG. 7 shows the protection effect of TJM2010-5 on IRI renal function of mice kidney (serum creatinine, urea nitrogen).

FIG. 8 is the flow chart showing that TJM2010-5 can reduce the T cell proliferation triggered by the CpG-stimulated DC activation.

FIG. 9a shows the real-time quantitative PCR analysis on reduction of inflammatory factor IL-1β in graft by TJM2010-5.

FIG. 9b shows the real-time quantitative PCR analysis on reduction of inflammatory factor TNF-α in graft by TJM2010-5.

FIG. 9c shows the real-time quantitative PCR analysis on reduction of inflammatory factor IL-6 in graft by TJM2010-5.

FIG. 10a shows the reduction of inflammatory cell infiltration in pulmonary inflammation model by TJM2010-5 (total number of cells in bronchial alveolar).

FIG. 10b shows the reduction of inflammatory cell infiltration in pulmonary inflammation model by TJM2010-5 (neutrophil count in bronchial alveolar).

FIG. 11a shows the comparison of reduction effects of TJM2010-5 on the activity of myeloperoxidase in lung tissue.

FIG. 11b shows the comparison of reduction effect of TJM2010-5 on concentration of interleukin-6 in lung tissue.

FIG. 12A shows the comparison of weights of mice obtained in the experiment of inhibiting colitis induced by AOM/DSS and preventing the colorectal cancer induced by AOM/DSS with TJM2010-5.

FIG. 12B shows the comparison of survival rates of mice obtained in the experiment of inhibiting colitis induced by AOM/DSS and preventing the colorectal cancer induced by AOM/DSS with TJM2010-5.

FIG. 12C shows the colon anatomy of mice obtained in the experiment of inhibiting colitis induced by AOM/DSS and preventing the colorectal cancer induced by AOM/DSS with TJM2010-5.

FIG. 12D shows the comparison among colorectal cancer incidence rates observed in the experiment of inhibiting colitis induced by AOM/DSS and preventing the colorectal cancer induced by AOM/DSS with TJM2010-5.

FIG. 12E shows the comparison among colon pathological sections of these groups used in the experiment of inhibiting colitis induced by AOM/DSS and preventing the colorectal cancer induced by AOM/DSS with TJM2010-5.

FIG. 13A shows the inhibitory effects of TJM2010-5 on activation of IRAK4.

FIG. 13B shows the inhibitory effects of TJM2010-5 on activation of NF-Bp65.

FIG. 13C shows the inhibitory effects of TJM2010-5 on expressions of various inflammatory factors, where FIG. 13C1-5 show the expressions of TNF-α, IL-6, G-CSF, MIP-1β, TGF-β1 respectively.

FIG. 14A shows the tissue sections comparison of the inhibitory effect of TJM2010-5 on inflammatory cells.

FIG. 14B shows comparison of inhibitory effect of TJM2010-5 on F4/80+CD11b+ tumor-associated macrophage in LPMCs.

FIG. 14C shows the comparison of inhibitory effect of TJM2010-5 on Gr-1^{high}CD11b^{high} MDSC myeloid suppressor cells infiltration.

FIG. 14D shows the effect of TJM2010-5 on reduction of IL-6 expression.

FIG. 15a shows the survival curve obtained in endoxemia lethal experiment.

FIG. 15b shows the survival curve obtained in septicopyemia lethal experiment.

### DETAILED DESCRIPTION OF THE INVENTION

The invention is further described in detail hereunder in accordance with figures and specific implementation methods.

### Application Example 1: Application of TJM2010-5 in Preventing Transplant Rejection and Inducing Transplantation Immune Tolerance

1) Application of TJM2010-5 in Mice Heart Transplantation Model

Four groups are set in the experiment, which are: control group (conducting heart transplantation without any treatment), CMC group (vehicle control group), TJM2010-5 treatment group (effect detection group) and ciclosporinA (CsA) control group. The specific treatment methods as follows:

Control group: Take and transplant the heart of Balb/c mice to the abdominal cavity of C57bl/6 mice, and no special treatment is provided after operation;

CMC control group: Take and transplant the heart of Balb/c mice to the abdominal cavity of C57bl/6 mice, and provide intraperitoneal injection of sodium carboxymethyl cellulose (0.5% CMC) solution without TJM2010-5 on day 0 to day 6 after the heart transplantation, and the dose is 200 μl.

TJM2010-5 treatment group: Take and transplant the heart of Balb/c mice to the abdominal cavity of C57bl/6 mice, and provide intraperitoneal injection of TJM2010-5 dissolved in CMC on day 0 to day 6 after the heart transplantation, and the dose is 150 mg/kg.

CsA treatment group: Take and transplant the heart of Balb/c mice to the abdominal cavity of C57bl/6 mice, and provide intraperitoneal injection of CsA on day 0 to day 6 after the heart transplantation, and the dose is 15 mg/kg/day.

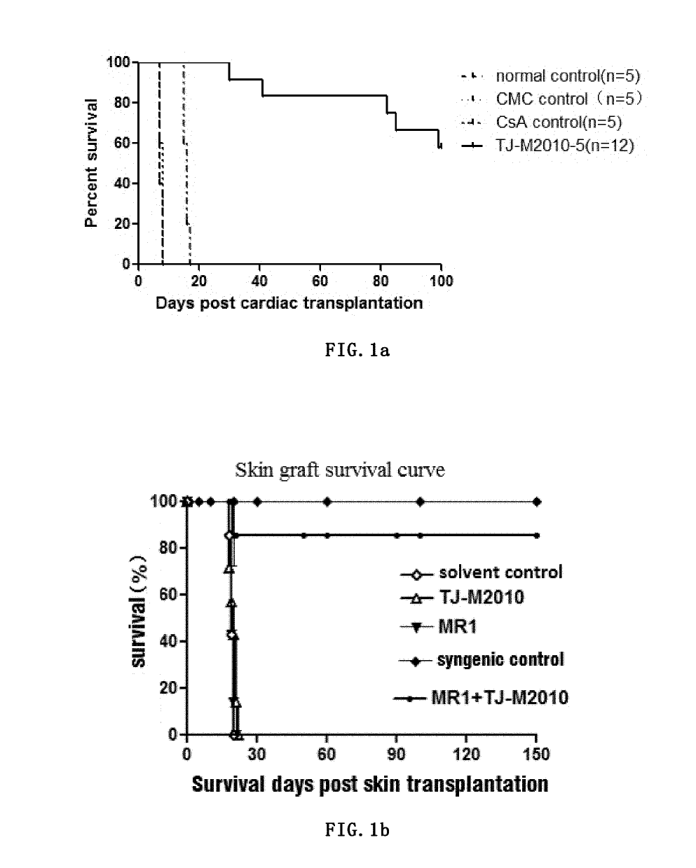

The experiment results are shown in the figure of survival curve (see FIG. 1a), where, the Balb/c and C57bl/6 in FIG. 1a represent two mouse strains, and the groups are: general control group in which the heart of Balb/c is transplanted to C57bl/6; CMC vehicle control group, C57bl/6 to C57bl/6 is the homologous comparison diagram; TJ-M2010 treatment group and CsA control group. The results show that the rejection time of control group is about 8 days, which is basically the same with that reported in literatures; there is no difference in CMC vehicle control group; and in TJM2010-5 treatment group, 80% of heart allograft can survive up to 100 days, which is obviously longer than that of control group.

2) Application of TJM2010-5 combined with costimulatory molecules inhibitor-anti-CD154 monoclonal antibody (MR1) in mice skin transplantation model

Five groups are set in the experiment, which are: allogeneic skin grafting control group (the rejection time reported in literature is 8-10 days), TJM2010-5 group, MR1 group and combination treatment group (TJM2010-5+MR1).

The specific handling methods as follows:

Allogeneic skin grafting CMC control group: take and transplant the skin of Balb/c mice to the back of C57bl/6 mice, and provide intraperitoneal injection of 0.5% CMC on day 0 to day 3, and day 5, 7, 9, 11, 13, 15 after the operation, the does is 200 μl/day; in the homogenic skin transplantation group: take and transplant the heart of C57bl/6 mice to the back of C57bl/6 mice, and no special treatment is provided after operation.

The TJM2010-5 Group: take and transplant the skin of Balb/c mice to the back of C57bl/6 mice, and provide intraperitoneal injection of the TJM2010-5 dissolved in 0.5% CMC on day 0 to day 3, and day 5, 7, 9, 11, 13, 15 after the operation, the does is 150 mg/kg/d;

The MR1 Group: take and transplant the skin of Balb/c mice to the back of C57bl/6 mice, and provide intraperitoneal injection of MR 1 on day 0, 1, 3, 7, 14 after the operation, the does is of 200 μg/d;

Combination treatment group: take and transplant the skin of Balb/c mice to the back of C57bl/6 mice, provide intraperitoneal injection of TJM2010-5 dissolved in 0.5% CMC on day 0 to day 3, and day 5, 7, 9, 11, 13, 15 after the operation, the dose is 150 mg/kg/d; provide intraperitoneal injection of MR1 on day 0 to day 3, and day 5, 7, 9, 11, 13, 15 after the operation, and the dose is 200 μg/d;

The experiment results are shown in the skin graft survival curve (see FIG. 1b). FIG. 1b shows the results of the solvent control group (allogeneic skin grafting control group, the rejection time reported in literature is 8-10 days), TJM2010-5 group, MR1 group and syngeneic control group (corresponding to the combination treatment group (TJM2010-5+MR1)), where the graft rejection time of the allogeneic skin grafting control group is about 10 days, which is consistent with that reported in literatures, and has no statistic difference, while in combination treatment group (TJM2010-5+MR1), the skin graft can survive up to 150 days (it is considered in the literature as graft tolerance when it is >100 days), which means that TJM2010-5 alone and MR1 alone has no obvious effect on tolerance induction, but the combination of TJM2010-5 and MR1 has significant effect, and can induce tolerance of the skin graft.

The experiment results mentioned above shows that MyD88 has significant effect in preventing post transplantation rejection and inducing immune tolerance, so it can be used as a special immune inhibitor, or transplantation tolerance inducer (short-term administration can let the skin graft with difficult to induce tolerance survives), transplantation tolerance maintenance agent (such as the ability to prevent the rejection induced by bacterial infection). For its unique roles, it cannot be compared and substituted by any current immune inhibitor.

### Application Example 2: Application of MyD88 Inhibitor TJM2010-5 in Treatment of GVHD

1) Experiment on Effect of TJM2010-5 in Treating GVHD

### Experimental Procedure

Construction of GVHD model: Apply myeloablativetotal body irradiation (TBI, 710cGy) (HI-ENERGY PRIMV S-M X-ray source) on female BALB/c (H-2d) mice, and inject the bone marrow cell of 1.0×10 of C57BL/6(B6, H-2^{b}) mice into it within 2 hours, and inject the B6spleen cells of 1.0×10¹⁰ into some of these female BALB/c (H-2d) mice at some time.

Groups: GVHD mice are divided into 4 groups, where the first group is control group, and the 8 mice in the group are provided with no post operation treatment; the second group is provided with intragastric administration of TJM2010-5 of 50 mg/kg/day since the day of irradiation; the third group is the MR1 group, in which the 8 mice are provided with the treatment of MR1 of 10 mg/kg/time on day 0, 1, 2, 3, 7 and 14 after irradiation; the forth group is the combination treatment group (TJM2010-5+MR1), in which the 8 mice are treated with TJM2010-5 and MR1 by the method mentioned above.

Results:

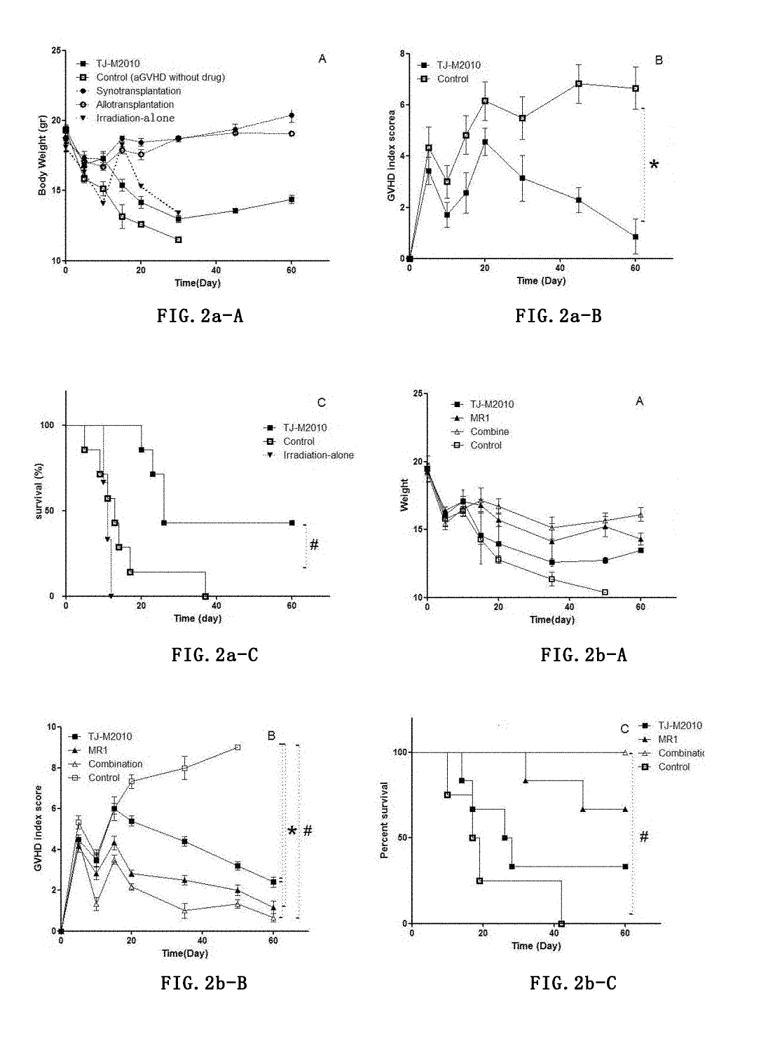

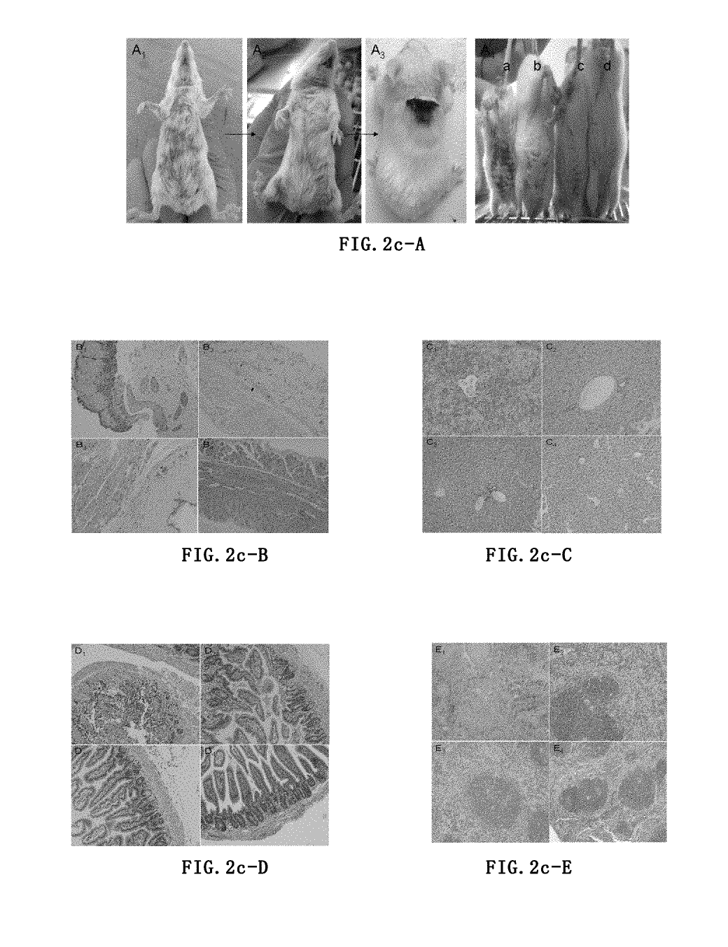

1. TJM2010-5 can significantly improve the survival rate and reduce GVHD level (see FIG. 2a): FIG. 2a shows the effect of TJM2010-5 in treatment of GVHD: where, FIG. 2a-A shows that the weight of TJM2010-5 treatment group is higher than that of control group. FIG. 2a-B shows that the GVHD level of TJM2010-5 treatment group is significantly lower than that of control group. And FIG. 2a-C shows that the survival rate of TJM2010-5 treatment group is significantly higher than that of control group. (P<0.05, P<0.05 and P<0.01)

2. The treatment combining TJM2010-5 and MR1 can completely reverse the acute GVHD in mice (see FIG. 2b). FIG. 2b shows that the curative effects of TJM2010-5 combined with MR1 on acute GVHD in mice: the curative effects of the combination treatment are manifested by the differences among weights (FIG. 2b-A), GVHD levels (FIG. 2b-B), survival rates (FIG. 2b-C) of treatment group and control groups (P>0.05: MR1 vs Combination, *P<0.05 and # P<0.01).

The curative effects of combination treatment is higher than that of single treatment of TJM2010-5 in terms of weight, GVHD level and survival rate, and the single treatment of MR1 is inefficient (P>0.05:MR1 vs Combination. *P<0.05 and # P<0.01).

The general performance and the tissue sections of mice of each group are significantly different from that of other group: FIG. 2c-B shows the comparison among skin sections of these groups, FIG. 2c-C shows the comparison among liver sections of these groups, FIG. 2c-D shows the comparison among intestine tissue section of these groups, FIG. 2c-E shows the comparison among spleen tissue sections of these groups (where, 1 refers to the control group, 2 refers to the single treatment group of TJM2010-5, 3 refers to the single treatment group of MR1, and 4 refers to the combination treatment group of TJM2010-Sand MR1). The GVHD mice are small and thin, with spare coat, severe diarrhea and high mortality, while the performances of TJM2010-5 treatment group are close to normal mice. The tissue sections show that each tissue has severe GVHD lesion, while in control group, the tissues have unobvious GVHD lesion or are normal.

Conclusion: TJM2010-5 can significantly inhibit GVHD, and it will have better, and can completely reverse GVHD when being combined with MR1.

2) Comparison experiment on effects of TJM2010-5 and TJM2010-1, TJM2010-2, TJM2010-3, TJM2010-4 in treating GVHD

The structures of TJM2010-1, TJM2010-2, TJM2010-3, TJM2010-4 medicaments are published in Chinese Patent Application 201110049579.7, and will not be described here.

The experimental procedure is the same with that of the experiment on effect of TJM2010-5 in treating GVHD: administrate one kind of TJM2010+MR1 combined medicament to each group of mice (8 mice), and provide the intragastric administration of TJM2010 of 50 mg/kg/day from the day of irradiation, and after 60 days, the survival rates of the mice in the groups corresponding to TJM2010-5 and TJM2010-1, TJM2010-2, TJM2010-3, TJM2010-4 are 100%, 28%, 16%, 37%, 6% respectively; the results show that TJM201-5 has unique effect on preventing graft-versus-host reaction (GVHD). The application of TJM2010-5 in treatment can significantly improve various symptoms of GVHD, and reverse 70% of GVHD. In case TJM2010-5 is combined with MR1, it can reverse 100% of GVHD. However, other aminothiazole compounds have no or little such curative effect.

### Application Example 3: Application of MyD88 Inhibitor in Treatment of Autoimmune Diseases

In vitro experiment—results of flow cytometer: prove that MyD88 inhibitor can prevent the maturity of DC, so it can be used to treat autoimmune diseases.

Procedure:

1. Derive the bone marrow cell from the BALB/c mice administrated with TJM2010-5, destruct the red blood cell, and place them into the RPMI1640 medium with the concentration of 2×10⁶/ML (adding GM-CSF of 10 ng/ml, IL-4 of 10 ng/ml);

2. Remove the suspension cell 48 hours later, and collect the suspension and anchorage-dependent cell on day 6.

3. Place DCs into 50 mM of TJM2010-5 for cultivating for 1 hour, add necrotic myocardium supernate, LPS (200 ng/ml), Poly I:C (20 mg/ml), CpG (10 mg/ml) to cultivate the cells for 12 hours.

4. Add the flow antibody FITC to label anti-CD80, CD86, and conduct detection with machine.

TJM2010-5 can inhibit the up-regulation of RAW264.7 cell on costimulatory molecules CD80 caused by TLR stimulant (LPS, CpG), which means that TJM2010-5 can effectively block the TLR signal pathway, thus to inhibit the cells' immune reaction.

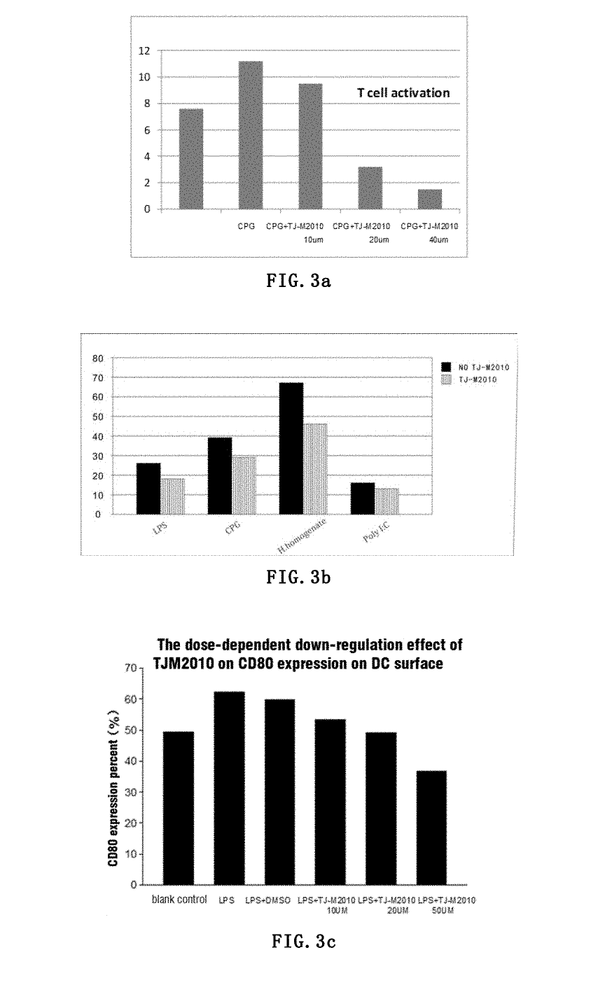

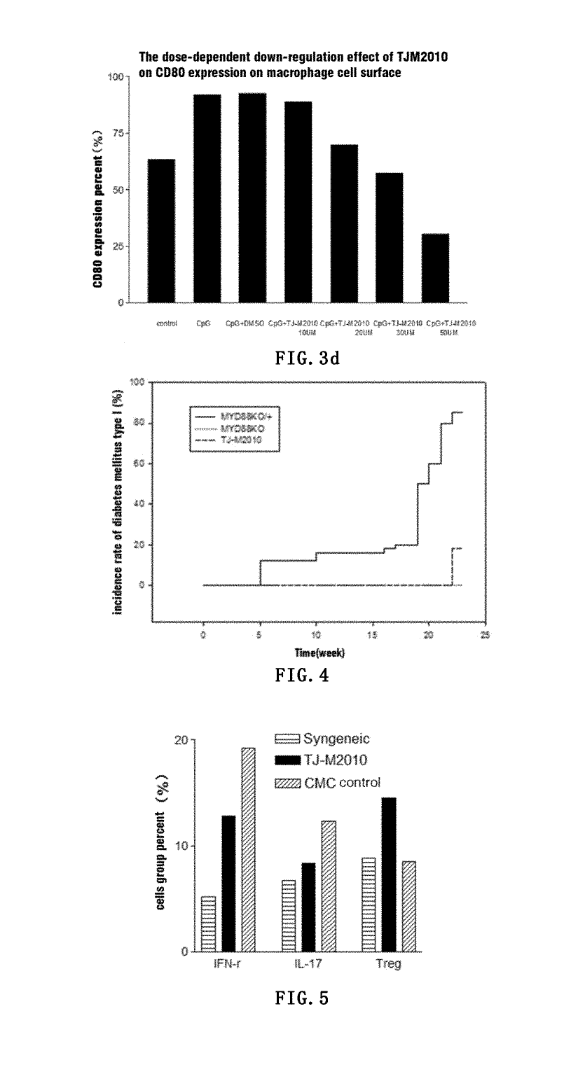

The experimental procedures of FIG. 3(c), (d) as follows:

Raw264.7: Place the cells into a 48-pore plate, the number of cells is 9×10⁵/pore, and 1 ml of medium system is added to each pore. Firstly, add TJM2010-5 of different concentration gradients to pre-incubate the cells for 2 h, then, add the CPG to get the final concentration of 40 ug/ml, and then incubate them in CO₂ incubator at 37° C. for one night (12 h). Add the flow antibody FITC to label anti-CD80, CD86, and conduct detection with machine;

DC: Place the cells into a 48-pore plate, the number of cells is 1×10⁶/pore, and 1 ml of medium system is added to each pore. Firstly, add TJM2010-5 of different concentration gradients to pre-incubate the cells for 2 h, then, add the LPS to get the final concentration of 1 ug/ml, and then incubate them in CO₂ incubator at 37° C. for one night (12 h).

Add the flow antibody FITC to label anti-CD80, CD86, and conduct detection with machine. The two figures mentioned above show that TJM2010-5 has a concentration-dependent inhibition on expression of CD80 on DC and macrophage surfaces. The results are shown in FIG. 3, where FIG. 3a shows that TJM2010 has a dose-dependent reduction effect on activation of T cells; FIG. 3b shows that LPS, CPG, cardiac tissue homogenate vehicle can stimulate DC, and TJM2010-5 can down-regulate the CD80 expression. FIG. 3c shows the dose-dependent down-regulation effect of TJM2010 on CD80 expression on DC surface, and FIG. 3d shows the dose-dependent down-regulation effect of TJM2010 on CD80 expression on macrophage cell surface. TJM2010-5 can inhibit up-regulation of costimulatory molecules CD80/CD86 incurred by LPS, CpG.

The detection results mentioned above show that MyD88 inhibitor can reduce CD80 expression, thus to prevent the maturity of DC cell.

It has been approved that the maturity of DC cells is the key step for development of various autoimmune diseases, such as autoimmune cardiomyopathy, experimental autoimmune uveitis, diabetes mellitus type I, multiple sclerosis and lupus erythematosus etc. Therefore, MyD88 inhibitor TJM2010-5 can be used for treatment of such diseases.

In vivo experiment: The effect of MyD88−/− and TJ-M2010 on construction of diabetes mellitus type I model

### Experimental Procedure

1. Experimental groups: MyD88KO NOD mice, MyD88KO/+NOD mice, NOD mice TJM2010-5 treatment group.

2. Treatment group: Provide intraperitoneal injection of TJM2010-5 dissolved in 0.5% CMC of 150 mg/kg/d on the day before antigen injection, and on day 0 to day 3, and day 5, 7, 9, 11, 13 and 15 day after injection.

3. Provide intraperitoneal injection of mycobacterium antigen to mice in each group and monitor the concentration constantly.

4. Feed in clean level and observe for 30 weeks, and take the tail venous blood to test the non-fasting blood-glucose. The standard for diabetes modeling is obtaining the blood-glucose no less than 22 mmol/L for 2 times successively.

The curve of incidence rate of diabetes mellitus type I is shown in FIG. 4:

The results show that in MyD88KO heterozygote group, the incidence rate of diabetes mellitus type I will increase gradually as time prolongs, while no diabetes is detected in MyD88KO homozygote group; the incidence rate of diabetes mellitus type I is equivalent to that of MyD88KO homozygote group, which means that MyD88 pathway has inevitable connection with diabetes mellitus type I, and the diabetes can be prevented by blocking its pathway. Therefore, small molecule MyD88 inhibitor TJ-M2010 can become the effective prevention and treatment method for diabetes mellitus type I.

### Application Example 4: Application of MyD88 Inhibitor in Prevention and Treatment of Ischemia Reperfusion Injury

In vitro experiment: conduct analysis on lymphocyte subpopulation in receptor spleen receiving stimulation of antigen (homogenic, allogeneic), and flow cytometer detection on CD4+CD25+Foxp3+T cell ratio in receptor receiving TJM2010-5 treatment.

### Experimental Procedure: TJM2010-5

1. Take the receptor spleen of different groups (homogenic, allogenic antigenic stimulation), and grind them to separate lymphocyte;

2. Add the IFN-γ labeled by flow antibody APC and IL-17 labeled by APC, CD25 labeled by APC and Foxp3 labeled by PE.

3. Analyze the lymphocyte subpopulation in receptor spleen of various groups, and the CD4+CD25+Foxp3+T cell ratio in receptor receiving TJM2010-5 treatment with flow cytometry.

As shown in FIG. 5, the results show that the CD4+CD25+Foxp3+T cell ratio in receptor receiving TJM2010-5 treatment was up-regulated significantly, and IFN-γ and IL-17 are significantly lower than that of CMC control group.

The results of analysis on lymphocyte subpopulation in receptor spleen of various groups and detection on the CD4+CD25+Foxp3+T cell ratio conducted after applying MyD88 inhibitor TJ-M2010 show that the applied TJM2010-5 can change the transplantation tolerance status of mice through up-regulating CD4+CD25+Foxp3+T cells. Lots of literatures show that the regulatory T cells can regulate the development of inflammation, the release of inflammatory factor, pro-inflammatory factors, and the cross-linking of cell factors during ischemia reperfusion through its immune inhibitory effect, thus to incur injury, therefore, the application of TJM2010-5 can inhibit the activation of NF-kB and reduce the expression of inflammatory factors (IFN-γ and IL-17) by inhibiting the TLR signal, thus to reduce injury.

### In Vivo Experiment

Experiment on reduction effects of MyD88 pathway blocking on renal ischemia reperfusion injury:

### Experimental Procedure

1. Groups: General C57bl/6 group (Control), CMC vehicle group, MYD88KO group, TJ-M2010 group, the 8 mice in each group will be provided with treatment of ischemia reperfusion: narcotize, block the left kidney with vascular clamp, and place them in incubator of 31 C° for 80 min; after that, open it and remove the right kidney, and then close the abdomen. Collect blood 24 hours later to conduct BUN, Cr detection.

2. For TJM2010-5 group and CMC group, provide intraperitoneal injection of TJ-M2010-5 dissolved in 0.5% CMC of 150 mg/kg/d on the day before and the day of operation; for CMC group: inject 0.5% CMC solution of 200 μl.

3. Observe the survival time of mice, and make the survival curve. Send the blood specimen to the pathology department for detection of BUN, Cr.

4. The results show that TJM2010-5 can significantly improve the survival rate after mice kidney IRI, and has a better protection effect on kidney functions.

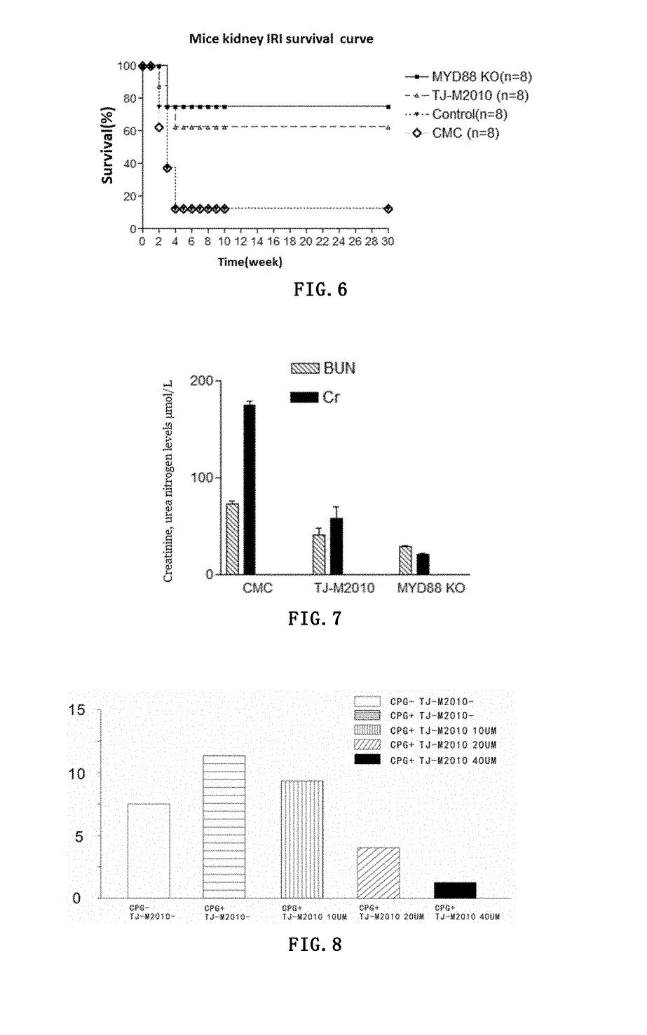

For the results, see FIG. 6 and FIG. 7. The experiments mentioned above show that the blocking of MyD88 pathway has significant protection effects against ischemia reperfusion injury, therefore, MyD88 inhibitor can be used to prevent and treat the post myocardial infarction ischemia reperfusion injury, post severed limb replantation ischemia reperfusion injury, and post transplantation ischemia reperfusion injury, as well as to play important roles in production of organ preservation solution, and cell preservation solution etc.

### Application Example 5: Application of MyD88 Inhibitor in Treatment of Inflammatory Diseases

In vitro experiment: Real time quantitative PCR Analysis on reduction effect of TJM2010-5 on T cell proliferation incurred by CpG stimulated DC activation and the inflammatory factors in graft

### Experimental Procedure

1. Take the thigh bone of Bal b/c mice, separate the bone marrow cell and add the GMS-CSF and IL-4 cytokines to cultivate marrow-derived DC.

2. Cultivate them until the sixth day, and dissociate the cells to remove immature DC. Conduct centrifugation and suspend them with 1640 medium.

3. Add mitomycin (to let the final concentration up to 50 μg/ml), and conduct water bath at 37° C. for 15 min Wash in 1640 medium and count.

4. Take the spleen of C57bl/6 mice, separate splenic lymphocytes with separating medium of mice lymphocytes and count.

5. Take the splenic lymphocytes of C57bl/6 mice and label CFSE.

6. Mix DC derived from Bal b/c and C57bl/6 mice lymphocytes for the mixed lymphocyte culture. And the grouped as follows:

Blank group: Do not add CPG and TJM2010-5 in the process of mixed lymphocyte culture.

Control group: Add CPG other than TJM2010-5 in the process of mixed lymphocyte culture.

Experiment group 1: Add CPG and TJM2010-5 in the process of mixed lymphocyte culture, where, the added TJM2010-5 is 10 μM.

Experiment group 2: Add CPG and TJM2010-5 in the process of mixed lymphocyte culture, where, the added TJM2010-5 is 20 μM.

Experiment group 3: Add CPG and TJM2010-5 in the process of mixed lymphocyte culture, where, the added TJM2010-5 is 40 μM.

1. Cultivate them until the third day, collect cells and detect the proliferation of C57bl/6 mice lymphopoiesis with flow cytometer.

The results of flow cytometer detection are shown in FIG. 8, which show that with the increasing of TJM2010-5, the proliferation in T cells (CD44 is its surface marker) reduces, which means that TJM2010-5 can reduce the T cell proliferation incurred by CpG stimulated Dc activation.

Procedures of Real Time Quantitative PCR:

1. Extract the total RNA from the receptor receiving antigen (homogenic, allogenic) stimulation by the TRIzol method;

2. Reversely transcribe them to cDNA by the two-step method of RT-PCR;

3. Make the standard curve to compare the measured relative level of IL-1β, TNF-α, IL-6.

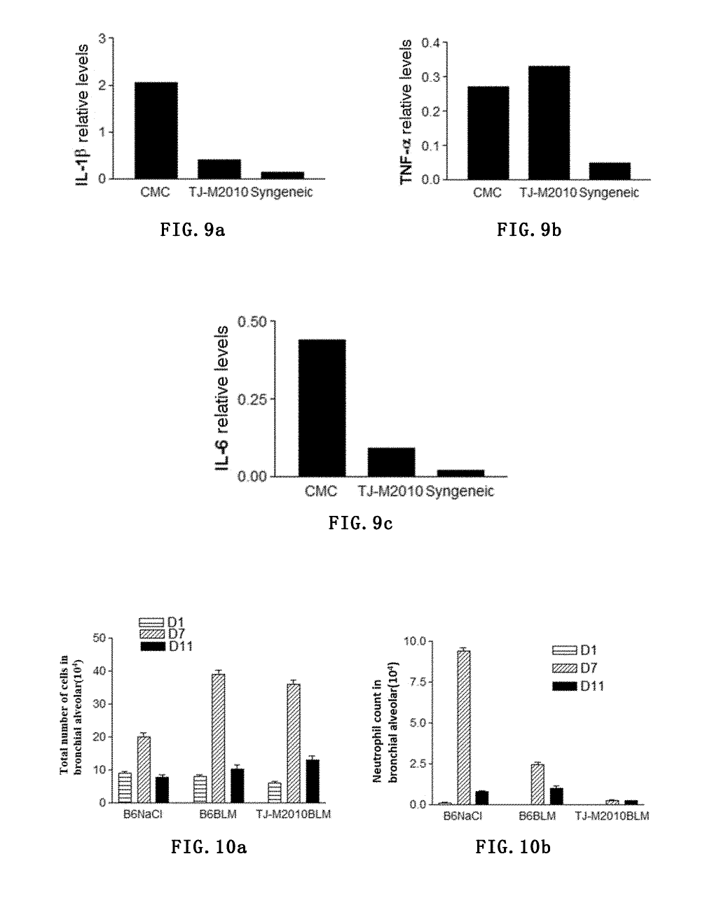

The results of real time quantitative PCR analysis on inflammatory factors (IL-1β, TNF-α, IL-6) in graft are shown in FIG. 9. The results show that the level of inflammatory factors in heat graft in TJM2010-5 group is significantly lower than that of control group, and has significant statistical difference. The results of in vitro experiments show that MyD88 inhibitor TJM2010-5 can effectively reduce the level of inflammatory factors in graft through blocking the pathway (the levels of IL-1β, IL-6 are significantly lower than those in CMC allogeneic transplantation group), which means that it has close relation with the generation of inflammatory factors, therefore, it can be used as the effective treatment method of various inflammations.

### In Vivo Experiment

Reduction of Inflammatory Reaction in Mice Trachea by MyD88 Pathway Blocking

Method and Procedure:

1. Animal grouping: C57bl/6(B6) NaCl (200 μl nasal drip) group, C57bl/6 (B6) BLM group and TJM2010-5BLM group (provide intraperitoneal injection of TJM2010-5 dissolved in 0.5% CMC of 150 mg/kg/d on day 0-3, 5, 7, 9, 11, 13, 15).

2. Construct pulmonary inflammation model with BLM (bleomycin) nasal drips: conduct airway anesthesia with 40 μl of ketaminexylazine, and administrate BLM sulfate by nasal dripping (300 μg or 15 mg/kg).

3. Collect cell and cytokines with bronchoalveolar lavage fluid (BAL): Cut open the trachea and insert the plastic sleeve; then lavage with 0.3 ml of PBS at 37° C., and withdraw the lavage fluid (the withdrawn fluid is more than 95%), and repeat the process for 10 times. The lavage fluid consists of two parts: one part is used for detection of cytokins (600 g, conduct centrifugation for 10 min, collect and store the supernatant at −80° C. for detection); the other part is used for cell counting (0.4 ml, including substratum, suspension), count at 4° C.

4. Detect cells and factors in tissue with pulmonary homogenate: After BAL, take and grind the whole lung, and conduct centrifugation to take supernatant, then, store it at −80 C° for detection of MPO.

5. Conduct activity detection of pulmonary MPO: Completely lavage the lung with saline passing through right heart, and take pulmonary tissue homogenate, then conduct centrifugation to take supernatant. After that, suspend and precipitate it with 1 ml of PBS (including 0.5% of HTAB, 5 mM of EDTA). Conduct centrifugation, add 50 μl of supernatant into tube (200 μl of PBS-HTAB-EDTA, 2 ml of HBSS, 100 μl of o-dianisidine dihydrochloride (1.25 mg/ml), 100 μl of H₂O₂ 0.05%), and put it into vortex water tank at 37° C. for 15 min. Then, stop the reaction with 100 μl of NaN₃ 1%, and detect the MPO absorption value with 460 nm.

6. Conduct cell counting: Dye with MG-1L for 4 min, and dye with 95% of GS-500 for 8 min. Then, make smear to count cells.

7. Conduct factor detection: detect the level of IL-6 with ELISA.

8. Conduct statistical analysis: U check and analyze statistical differences.

FIG. 9 shows that in the development of bronchitis of MYD88−/− mice, the recruitment of neutrophile granulocyte and lymphocyte reduces.

Experimental grouping: B6NaCL group, B6BLM group and TJ-M2010BLM group (n=4). FIG. 10 shows that in TJM2010-5 group, the recruitment of bronchialveolar neutrophile granulocyte reduces significantly. Where, Figure a shows the cell numbers on day 1, 7, 11, which show that there are statistical differences between WT mice and TJM2010-5BLM group; Figure b shows that bronchoalveolar neutrophil cells of WT mice reached the peak at 24 h, which will lasts for 7 days, and recover after 11 days, while its recruitment in TJM2010-5 group reduces significantly.

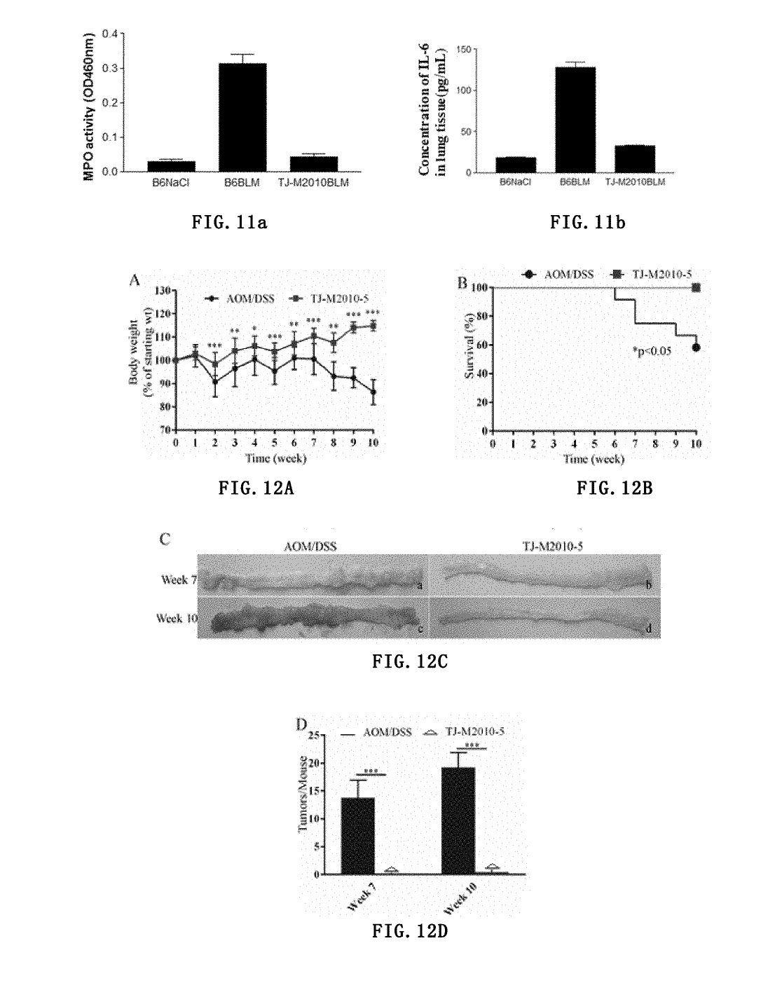

FIG. 11 shows that in TJM2010-5BLM group, the lung inflammation reaction induced by BLM reduces, which manifests as reduction of inflammatory cells and inflammatory factors. Figure a shows that the MPO factor in lung tissue (detection on day 7) reduced; Figure b shows that the level of IL-6 in lung tissue reduces at 24 h.

The experiments on recruitment of inflammatory cells and release of inflammatory factors show that in TJM2010-5BLM group, the lung inflammatory reaction reduces significantly, which proves the anti-inflammatory effects on TJM2010-5BLM.

The experiments mentioned above show that the blocking of MyD88 pathway can reduce the inflammatory reaction. Therefore, MyD88 inhibitor can be used to treat various chronic inflammatory diseases, such as chronic inflammatory bowel disease, asthma etc.

### Application Example 6: Application of MyD88 Inhibitor TJM2010-5 in Treatment of Chronic Inflammation, Inflammation-Associated Tumor and its Mechanism

1) Experiment on effect of TJM2010-5 in prevention and treatment of colitis and colorectal cancer

### Experimental Procedure

Construct the chronic colitis and colitis-associated colorectal cancer (CAC) model: apply intraperitoneal injection of 10 mg/Kg azoxymethane (AOM) (Sigma-Aldrich Chemical) to 40Balb/c mice, and start to execute three cycles of feeding of drinking water containing 2.5% of dextran sodium sulfate (DSS) (MP Biomedicals) 7 days later, and each cycle consists one week of feeding DSS water and two weeks of feeding common fresh water. Then, observe them in the following 10 weeks. The results show that the mice receiving AOM/DSS treatment all have colitis, with the symptoms of hemafecia, significant weight loss, and their survival rates are obviously lower than those of M2010-5 treatment group. 100% of mice in this group have colorectal cancer.

TJM2010-5 treatment group: Provide intragastric administration of TJM2010-5 of 50 mg/kg to 20 Balb/c mice in two days before providing the treatment mentioned above, and observe them in ten weeks.

Mechanism study: Take the specimen of serum, colon, and spleen etc. from 4 mice in each group 2 weeks and 7 weeks later to detect the infiltration of IRAK4 and inflammatory cytokines, macrophage and Gr-1^{high}CD11b^{high} MDSC, and the proportion of LPMCs (lamina propria mononuclearcells) F4/80⁺CD11b⁺ macrophage etc.

### Experimental Results

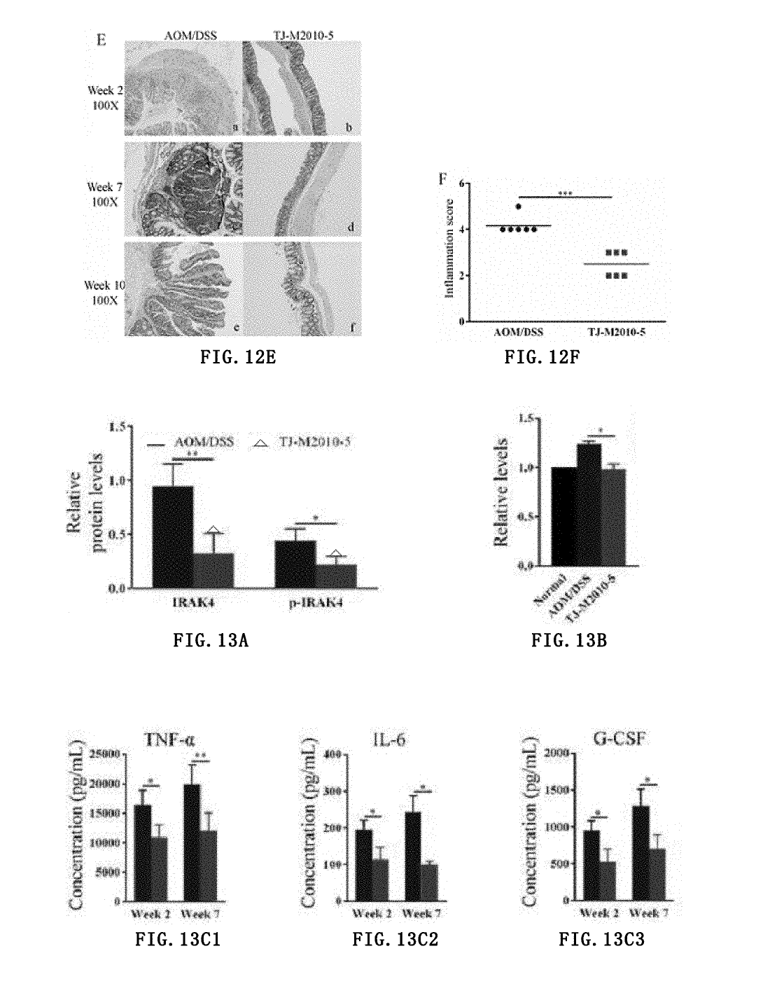

1. The weighs of all mice in TJM2010-5 treatment group reduces, and FIG. 12A shows that the weights of TJM2010-5 treatment group is obviously higher than that of untreated group.

2. The survival rate of TJM2010-5 treatment group is obviously higher than that of control group, as shown in FIG. 12B.

3. No apparent colitis is discovered in TJM2010-5 treatment group, and FIG. 12C shows that no mice in TJM2010-5 treatment group has colitis and colorectal cancer. See FIG. 12C, D, E,

Conclusion: MyD88 inhibitor TJM2010-5 can significantly inhibit AOM/DSS-induced colitis, and completely prevent the colorectal cancer induced by colitis.

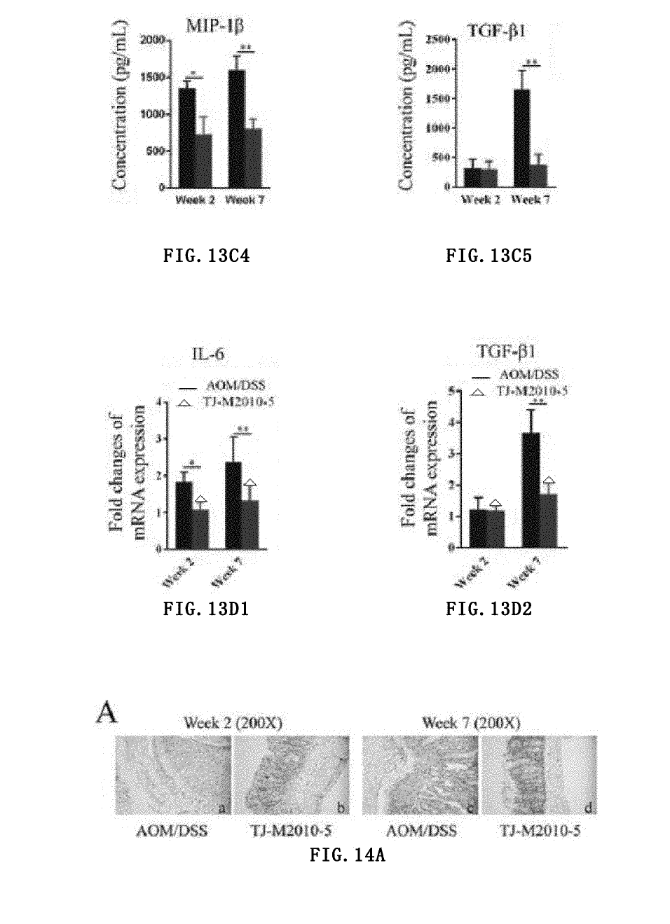

4. TJM2010-5 can inhibit the activation of IRAK4 and the generation of cytokines etc., see FIG. 13. FIG. 13A shows that TJM2010-5 can inhibit the activation of IRAK4, and reduce p-IRAK4, while the corresponding indexes of AOM/DSS in control group are obviously higher than those of treatment group. FIG. 13B shows that TJM2010-5 can inhibit the activation of NF-□Bp65. FIG. 13C shows that the generations of various inflammatory cytokines, such as TNF-α, IL-6, G-CSF, MIP-1β and TGF-β1 etc. in TJM2010-5 treatment group are obviously lower than those of control group. And FIG. 13D shows that the levels of IL-6 and TGF-β1 mRNA in TJM2010-5 treatment group is obviously lower than those of control group.

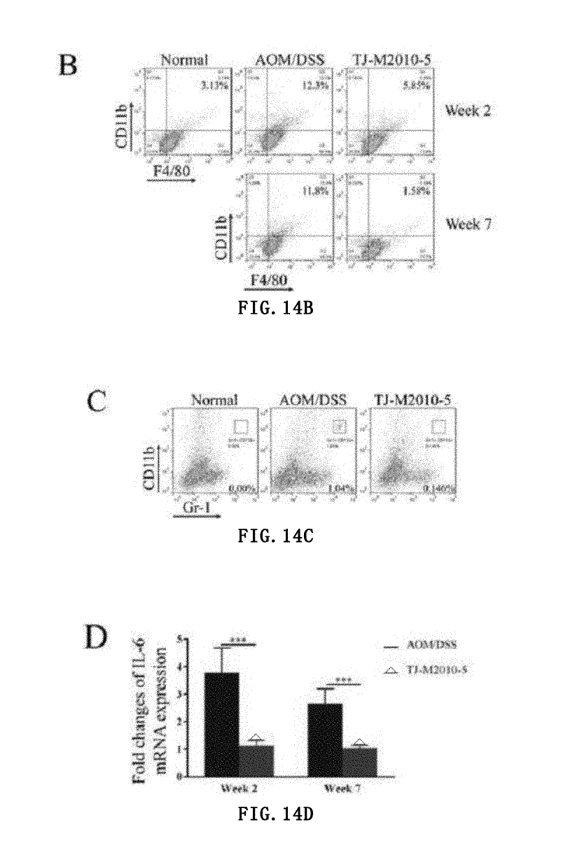

5. Treatment with TJM2010-5 can reduce the infiltration of macrophage and Gr-1^{high}CD11b^{high} MDSC (myeloid suppressor cells) and the expression of IL-6. The F4/80+CD11b+tumor-associated macrophage in LPMCs reduces, see FIG. 14.

2) Comparison experiment on effects of TJM2010-5 and TJM2010-1, TJM2010-2, TJM2010-3, TJM2010-4 in prevention and treatment of colitis and colorectal cancer

The experimental procedure is the same with that of the experiment on effect of TJM2010-5 in preventing and treating colitis and colorectal cancer: Apply intragastric administration of TJM2010-1, TJM2010-2, TJM2010-3, TJM2010-4, TJM2010-5 of 50 mg/kg to 20 Balb/c mice in each group of totally 6 groups, and then, observe them in the following 10 weeks. The mice with colitis and colorectal cancer in the group corresponding to TJM2010-5 and TJM2010-1, TJM2010-2, TJM2010-3, TJM2010-4, TJM2010-5 accounts 0%, 71.52%, 42.28%, 60.19%, 81.34% respectively; 2. TJM2010-5 can 100% prevent and cure chronic colitis and prevent the colorectal cancer incurred by chronic colitis. Intragastric administration of TJM2010-5 can make the above symptoms disappear, and the pathological test proves that there is no apparent colitis expression. Other aminothiazole compound has no or little such curative effect.

### Application Example 7: Application of MyD88 Inhibitor in Treatment of Endoxemia and Septicopyemia

Part one: Observe the effect of MyD88 inhibitor on mortality of mice with endoxemia. The mice will be divided into 2 groups randomly:

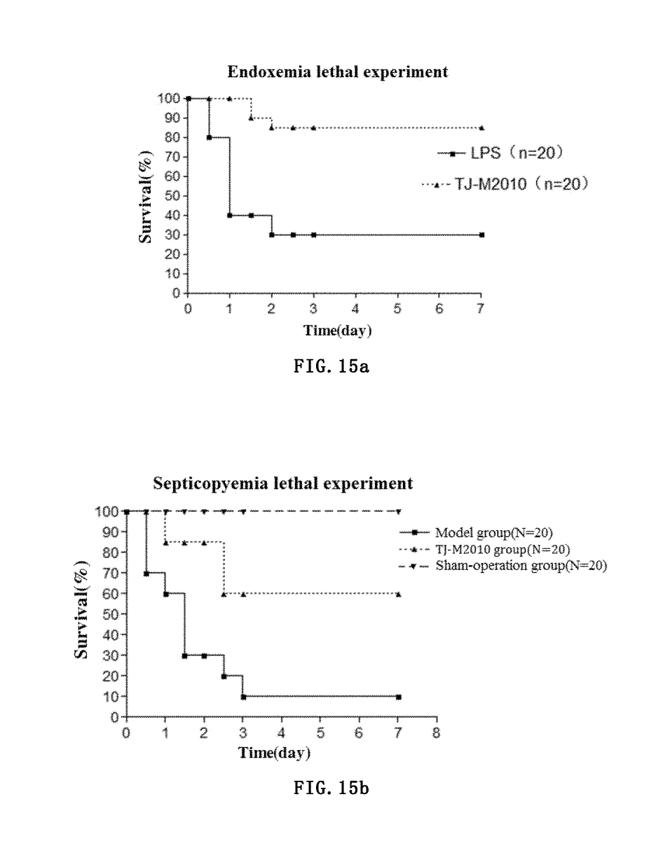

Vehicle control group and experiment group (TJM2010-5 treatment group), and 20 mice in each group. Where, the TJM2010-5 treatment group is provided with the intragastric administration of TJM2010-5 (0.5% CMC solvent, 25 mg/ml) at the dose of 250 mg/kg (200 μL per mouse); the vehicle control group is provided with the intragastric administration of 0.5% CMC, 200 μL per mouse. Apply the intragastric administration once a day, and for three days successively. And on day 3, after the intraperitoneal injection, provide the intraperitoneal injection of LPS 1 hour later, then, observe the survival condition every twelve hours, and for three days. The observed survival curve is shown in FIG. 15(a), and as shown in the figure, the two MyD88 inhibitors used can effectively delay the death incurred by endotoxin, and reduce the lethality of endotoxin.

Part two: Observe the effect of MyD88 inhibitor on mortality of mice with pyohemia. The mice will be randomly divided into: sham-operation group, model group, MyD88 inhibitor treatment group. Except for the mice in sham-operation group that only receive post-operative suture, the mice in pyohemia model group and MyD88 inhibitor treatment group are provided with the cecal ligation and puncture (CLP), to copy the pyohemia mice model. At 1 h after operation, apply intragastric administration of 0.5% CMC 200 μL or TJM2010-5 of 250 mg/kg (200 μL) for 4 times successively, and each time lasts for 12 h. After operation, observe the survival rate of mice in each group every 12 h, and for 72 h successively. The observed survival rate is shown in FIG. 15(b).

As shown in the figure, MyD88 inhibitor TJM2010-5 can significantly improve the death time and final mortality of pyohemia in mice.

1-6. (canceled)

7. A method for treating various inflammations, chronic inflammatory diseases and the related inflammation-associated tumors, comprising administering an effective amount of TJM2010-5 of formula: 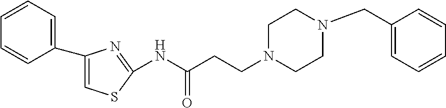 [SMILES: O=C(CCN1CCN(Cc2ccccc2)CC1)Nc1nc(-c2ccccc2)cs1]
to a human or animal in need thereof.

8. A method for treating various autoimmune diseases, comprising administering an effective amount of TJM2010-5 of formula: 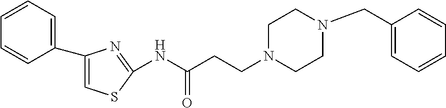 [SMILES: O=C(CCN1CCN(Cc2ccccc2)CC1)Nc1nc(-c2ccccc2)cs1]
to a human or animal in need thereof.

9. A method for treating ischemia reperfusion injury, comprising administering an effective amount of TJM2010-5 of formula: 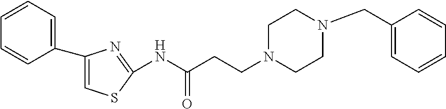 [SMILES: O=C(CCN1CCN(Cc2ccccc2)CC1)Nc1nc(-c2ccccc2)cs1]
to a human or animal in need thereof.
